(19) United States
(12) Patent Application Publication
(10) Pub. No.: US 2025/0032697 A1
(43) Pub. Date: Jan. 30, 2025

(54) SYSTEMS AND METHODS FOR CONTROLLING PRESSURE DELIVERY BY NEGATIVE PRESSURE WOUND THERAPY DEVICES

(71) Applicant: T.J.Smith and Nephew,Limited, Hull (GB)

(72) Inventors: Ben Alan Askem, Leeds (GB); David Michael Elder, Hull (GB); Mark John Ellerington, Hull (GB); Benjamin James Gardner, Hull (GB); Edward Yerbury Hartwell, Hull (GB); Reece James Knight, Kingston upon Hull (GB); Andrea Maggiore, Hull (GB); Natasha Rose Middleton, Hull (GB); Samuel John Mortimer, Kingston upon Hull (GB); Fraser George Weedon, Hull (GB); Amy Nicole Wheldrake, Bristol (GB)

(21) Appl. No.: 18/716,925

(22) PCT Filed: Dec. 13, 2022
(86) PCT No.: PCT/EP2022/085556

§ 371 Date: Jun. 5, 2024

(30) Foreign Application Priority Data
Dec. 21, 2021 (GB) 2118591.3

(51) Int. Cl. A61M 1/00

(52) U.S. Cl. CPC A61M 1/96; A61M 2205/3334; A61M 2205/502

(57) ABSTRACT

A negative pressure wound therapy apparatus can include a housing, a negative pressure source supported by the housing and configured to aspirate fluid from a wound covered by a wound dressing, a switch positioned on an exterior surface of the housing, and a controller configured to operate the negative pressure source. The controller can be configured to, responsive to receiving a first user input via the switch, operate the negative pressure source to aspirate fluid from the wound at a first fluid flow rate. The controller can be configured to, responsive to receiving a second user input via the switch subsequent to receiving the first user input, operate the negative pressure source to aspirate fluid from the wound at a second fluid flow rate greater than the first fluid flow rate.

### TECHNICAL FIELD

Embodiments described herein relate to apparatuses, systems, and methods for the treatment of wounds, for example using dressings in combination with negative pressure wound therapy.

### DESCRIPTION OF THE RELATED ART

Many different types of wound dressings are known for aiding in the healing process of a human or animal. These different types of wound dressings include many different types of materials and layers, for example, gauze, pads, foam pads or multi-layer wound dressings. Topical negative pressure (TNP) therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds, and abdominal wounds or the like. TNP therapy assists in the closure and healing of wounds by reducing tissue edema, encouraging blood flow, stimulating the formation of granulation tissue, removing excess exudates and may reduce bacterial load. Thus, reducing infection to the wound. Furthermore, TNP therapy permits less outside disturbance of the wound and promotes more rapid healing.

### SUMMARY

A negative pressure wound therapy apparatus can include a housing. The apparatus can include at least one negative pressure source supported by the housing, the at least one negative pressure source configured to aspirate, via a fluid flow path, fluid from a wound covered by a wound dressing. The apparatus can include a switch positioned on an exterior surface of the housing. The apparatus can include a controller configured to operate the at least one negative pressure source. The controller can be configured to, responsive to receiving a first user input via the switch, operate the at least one negative pressure source to aspirate fluid from the wound at a first fluid flow rate. The controller can be configured to, responsive to receiving a second user input via the switch subsequent to receiving the first user input, operate the at least one negative pressure source to aspirate fluid from the wound at a second fluid flow rate greater than the first fluid flow rate.

The negative pressure wound therapy apparatus of any of the preceding paragraphs and/or any of the apparatuses, systems, or devices disclosed herein can include one or more of the following features. The switch can be the only switch positioned on the exterior surface of the housing and configured to activate and deactivate provision of negative pressure wound therapy. The controller can be configured to, responsive to receiving a third user input via the switch subsequent to receiving the second user input, operate the at least one negative pressure source to aspirate fluid from the wound at a third fluid flow rate greater than the second fluid flow rate. The controller can be configured to operate the at least one negative pressure source to aspirate the fluid at the first, second, or third fluid flow rate during establishing a target negative pressure level at the wound. The controller can be configured to, subsequent to establishing a target negative pressure level at the wound and responsive to receiving a fourth user input via the switch, deactivate the at least one negative pressure source to pause aspiration of fluid from the wound.

The negative pressure wound therapy apparatus of any of the preceding paragraphs and/or any of the apparatuses, systems, or devices disclosed herein can include one or more of the following features. The at least one negative pressure source can include an actuator, and the controller can be configured to overdrive the actuator to operate the at least one negative pressure source to aspirate fluid from the wound at the second or third fluid flow rate. The first fluid flow rate can correspond to a default fluid flow rate for establishing a target negative pressure level at the wound following initiation of negative pressure wound therapy. The at least one negative pressure source can include first and second negative pressure sources, the first negative pressure source configured to aspirate fluid from the wound at the first fluid flow rate. The second negative pressure source can be configured to be activated by the controller responsive to the second or third user input to assist the first negative pressure source to aspirate fluid from the wound at the second or third fluid flow rate. The switch can include a button. The apparatus can include another switch configured to activate and pause provision of negative pressure wound therapy. The apparatus can be a canisterless apparatus.

A negative pressure wound therapy apparatus can include a housing. The apparatus can include a negative pressure source supported by the housing, the negative pressure source configured to provide, via a fluid flow path, negative pressure to a wound covered by a wound dressing. The apparatus can include a controller configured to operate the negative pressure source. The controller can be configured to, responsive to receiving a first user input, operate the negative pressure source to provide negative pressure to the wound at a first negative pressure rate. The controller can be configured to, responsive to receiving a second user input subsequent to the first user input, operate the negative pressure source to provide negative pressure to the wound at a second negative pressure rate lower than the first negative pressure rate.

The negative pressure wound therapy apparatus of any of the preceding paragraphs and/or any of the apparatuses, systems, or devices disclosed herein can include one or more of the following features. The apparatus can include a switch positioned on an exterior surface of the housing, wherein the first and second user inputs can be received via the switch. The switch can be the only switch positioned on the exterior surface of the housing and configured to activate and deactivate provision of negative pressure wound therapy. The switch can include a rotary switch configured to be set by the user to a position of a plurality of positions associated with a plurality of second negative pressure rates. The controller can be configured to operate the negative pressure source to provide negative pressure to the wound at the first or second negative pressure rate during establishing a target negative pressure level at the wound. The controller can be configured to, responsive to a determination that 1) a duration of time during which the negative pressure source has been providing negative pressure to the wound at the second negative pressure satisfies a threshold and 2) a target negative pressure level has not been established at the wound, operate the negative pressure source to provide negative pressure to the wound at the first negative pressure rate. The controller can disable the switch responsive to the determination. The first negative pressure rate can correspond to a default negative pressure rate for establishing a target negative pressure level at the wound following initiation of negative pressure wound therapy.

Disclosed herein are methods of operating any of the negative pressure wound therapy apparatuses of any of the preceding paragraphs and/or any of the devices, apparatuses, or systems disclosed herein.

Disclosed herein are kits that include the negative pressure wound therapy device of any of the preceding paragraphs and/or any of the devices, apparatuses, or systems disclosed herein and one or more wound dressings and/or a canister.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIG. 1A illustrates a negative pressure wound therapy system.

FIG. 1B illustrates another negative pressure wound therapy system.

FIG. 2A is an isometric view of a negative pressure wound therapy device and canister, showing the canister detached from the pump assembly of the device.

FIG. 2B is a back view of the negative pressure wound therapy device shown in FIG. 2A.

FIG. 2C illustrates a top surface of the negative pressure wound therapy device shown in FIG. 2A, showing a user interface.

FIG. 3 illustrates a schematic of a control system of a negative pressure wound therapy device.

FIG. 4 illustrates another negative pressure wound therapy system.

FIGS. 5A, 5B, and 5C illustrate a negative pressure wound therapy device.

FIG. 6 illustrates a graph of increasing the flow rate during delivery of negative pressure wound therapy.

FIG. 7 illustrates a dual pump system for increasing the flow rate during delivery of negative pressure wound therapy.

FIG. 8 illustrates a graph of establishing a negative pressure set point during delivery of negative pressure wound therapy.

### DETAILED DESCRIPTION

Embodiments disclosed herein relate to systems and methods of treating and/or monitoring a wound. Some embodiments of the negative pressure wound therapy devices disclosed herein can include a negative pressure source configured to be connected and/or fluidically coupled, via a fluid flow path, to a wound covered by a wound dressing and provide negative pressure to a wound.

Throughout this specification reference is made to a wound. The term wound is to be broadly construed and encompasses open and closed wounds in which skin is torn, cut or punctured or where trauma causes a contusion, or any other superficial or other conditions or imperfections on the skin of a patient or otherwise that benefit from pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

Embodiments of systems and methods disclosed herein can be used with topical negative pressure (“TNP”) or reduced pressure therapy systems. Briefly, negative pressure wound therapy assists in the closure and healing of many forms of “hard to heal” wounds by reducing tissue oedema, encouraging blood flow and granular tissue formation, or removing excess exudate and can reduce bacterial load (and thus infection risk). In addition, the therapy allows for less disturbance of a wound leading to more rapid healing. TNP therapy systems can also assist in the healing of surgically closed wounds by removing fluid. TNP therapy can help to stabilize the tissue in the apposed position of closure. A further beneficial use of TNP therapy can be found in grafts and flaps where removal of excess fluid is important and close proximity of the graft to tissue is required in order to ensure tissue viability.

As used herein, reduced or negative pressure levels, such as-X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects pressure that is X mmHg below 760 mmHg or, in other words, a pressure of (760−X) mmHg. In addition, negative pressure that is “less” or “smaller” than X mmHg corresponds to pressure that is closer to atmospheric pressure (for example, −40 mmHg is less than-60 mmHg). Negative pressure that is “more” or “greater” than-X mmHg corresponds to pressure that is further from atmospheric pressure (for example, −80 mmHg is more than-60 mmHg). In some cases, local ambient atmospheric pressure is used as a reference point, and such local atmospheric pressure may not necessarily be, for example, 760 mmHg.

Systems and methods disclosed herein can be used with other types of treatment in addition to or instead of reduced pressure therapy, such as irrigation, ultrasound, heat or cold, neuro stimulation, or the like. In some cases, disclosed systems and methods can be used for wound monitoring without application of additional therapy. Systems and methods disclosed herein can be used in conjunction with a dressing, including with compression dressing, reduced pressure dressing, or the like.

A healthcare provider, such as a clinician, nurse, or the like, can provide a TNP prescription specifying, for example, the pressure level or time of application. However, the healing process is different for each patient and the prescription may affect the healing process in a way the clinician or healthcare provider did not expect at the time of devising the prescription. A healthcare provider may try to adjust the prescription as the wound heals (or does not heal), but such process may require various appointments that can be time consuming and repetitive. Embodiments disclosed herein provide systems, devices, or methods of efficiently adjusting TNP prescriptions and delivering effective TNP therapy.

### Wound Therapy System

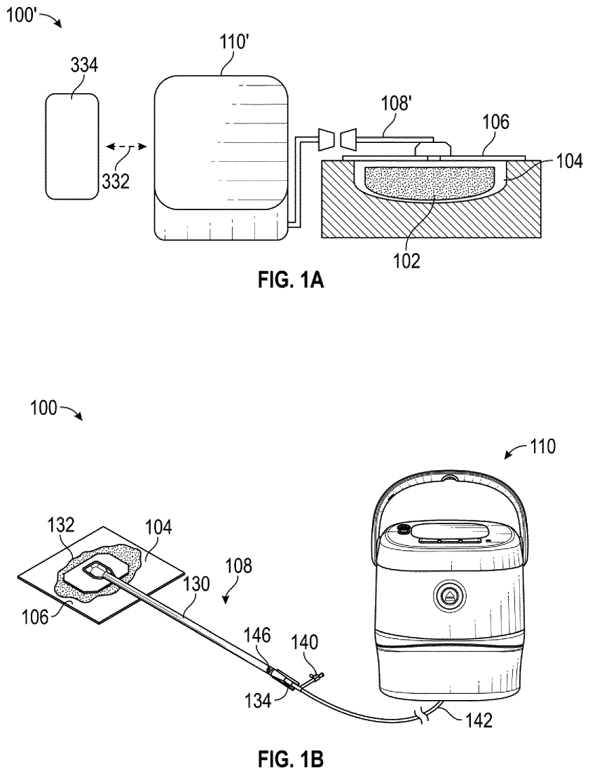

FIG. 1A schematically illustrates a negative pressure wound treatment system 100′ (sometimes referred to as a reduced or negative pressure wound therapy system, a TNP system, or a wound treatment system). In any implementations disclosed herein, though not required, the negative pressure wound treatment system 100′ can include a wound filler 102 placed on or inside a wound 104 (which may be a cavity). The wound 104 can be sealed by a wound cover 106, which can be a drape, such that the wound cover 106 can be in fluidic communication with the wound 104. The wound filler 102 in combination with the wound cover 106 can be referred to as a wound dressing. A tube or conduit 108′ (also referred to herein as a flexible suction adapter or a fluidic connector) can be used to connect the wound cover 106 with a wound therapy device 110′ (sometimes as a whole or partially referred to as a “pump assembly”) configured to supply reduced or negative pressure. The conduit 108′ can be a single or multi lumen tube. A connector can be used to removably and selectively couple a conduit or tube of the device 100′ with the conduit 108′.

In any of the systems disclosed herein, a wound therapy device can be canisterless, wherein, for example and without limitation, wound exudate is collected in the wound dressing or is transferred via a conduit for collection at another location. However, any of the wound therapy devices disclosed herein can include or support a canister.

Additionally, with any of the wound therapy systems disclosed herein, any of the wound therapy devices can be mounted to or supported by the wound dressing or adjacent to the wound dressing. The wound filler 102 can be any suitable type, such as hydrophilic or hydrophobic foam, gauze, inflatable bag, and so on. The wound filler 102 can be conformable to the wound 104 such that the wound filler 102 substantially fills the cavity of the wound 104. The wound cover 106 can provide a substantially fluid impermeable seal over the wound 104. The wound cover 106 can have a top side and a bottom side. The bottom side can adhesively (or in any other suitable manner) seal with the wound 104, for example by sealing with the skin around the wound 104. The conduit 108 or any other conduit disclosed herein can be formed from polyurethane, PVC, nylon, polyethylene, silicone, or any other suitable material.

The wound cover 106 can have a port (not shown) configured to receive an end of the conduit 108. In some cases, the conduit 108 can otherwise pass through or under the wound cover 106 to supply reduced pressure to the wound 104 so as to maintain a desired level of reduced pressure in the wound 104. The conduit 108 can be any suitable article configured to provide at least a substantially sealed fluid flow pathway or path between the wound therapy device 110′ and the wound cover 106, so as to supply the reduced pressure provided by the wound therapy device 110′ to wound 104.

The wound cover 106 and the wound filler 102 can be provided as a single article or an integrated single unit. In some cases, no wound filler is provided and the wound cover by itself may be considered the wound dressing. The wound dressing can then be connected, via the conduit 108, to a source of negative pressure of the wound therapy device 110′. In some cases, though not required, the wound therapy device 110′ can be miniaturized and portable, although larger conventional negative pressure sources (or pumps) can also be used.

The wound cover 106 can be located over a wound site to be treated. The wound cover 106 can form a substantially sealed cavity or enclosure over the wound. The wound cover 106 can have a film having a high water vapour permeability to enable the evaporation of surplus fluid, and can have a superabsorbing material contained therein to safely absorb wound exudate. In some cases, the components of the TNP systems described herein can be particularly suited for incisional wounds that exude a small amount of wound exudate.

The wound therapy device 110′ can operate with or without the use of an exudate canister. In some cases, as is illustrated, the wound therapy device 110′ can include an exudate canister. In some cases, configuring the wound therapy device 110′ and conduit 108′ so that the conduit 108′ can be quickly and easily removed from the wound therapy device 110′ can facilitate or improve the process of wound dressing or pump changes, if necessary. Any of the pump assemblies disclosed herein can have any suitable connection between the conduit 108′ and the pump.

The wound therapy device 110′ can deliver negative pressure of approximately −80 mmHg, or between about −20 mmHg and −200 mmHg. Note that these pressures are relative to normal ambient atmospheric pressure thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the wound therapy device 110′.

As will be described in greater detail below, the negative pressure wound treatment system 100′ can be configured to provide a connection 332 to a separate or remote computing device 334. The connection 332 can be wired or wireless (such as, Bluetooth, Bluetooth low energy (BLE), Near-Field Communication (NFC), WiFi, or cellular). The remote computing device 334 can be a smartphone, a tablet, a laptop or another standalone computer, a server (such as, a cloud server), another pump device, or the like.

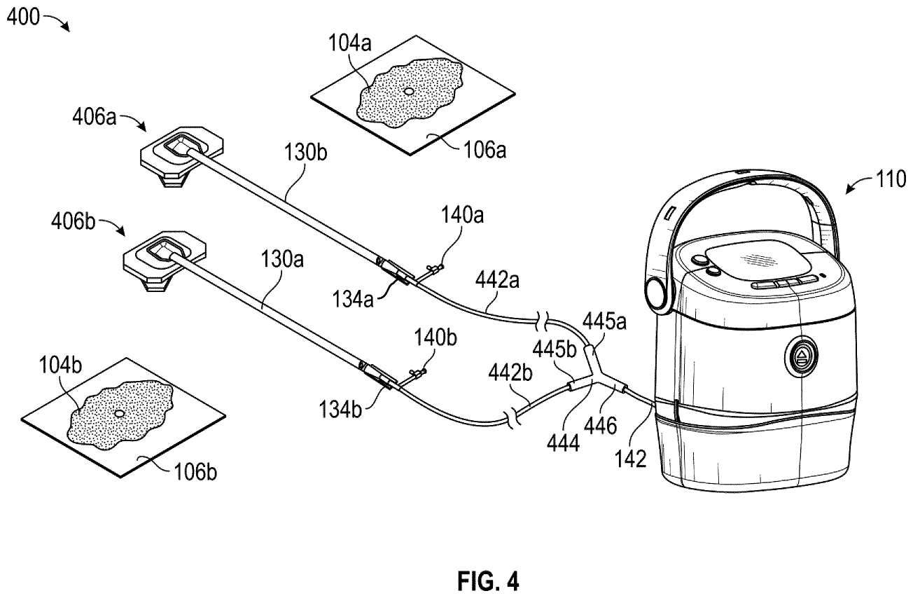

FIG. 1B illustrates another negative pressure wound treatment system 100. The negative pressure wound treatment system 100 can have any of the components, features, or other details of any of the other negative pressure wound treatment system disclosed herein, including without limitation the negative pressure wound treatment system 100′ illustrated in FIG. 1A or the negative pressure wound treatment system 400 illustrated in FIG. 4, in combination with or in place of any of the components, features, or other details of the negative pressure wound treatment system 100 shown in FIG. 1B and/or described herein. The negative pressure wound treatment system 100 can have a wound cover 106 over a wound 104 that can seal the wound 104. A conduit 108, such as a single or multi lumen tube can be used to connect the wound cover 106 with a wound therapy device 110 (sometimes as a whole or partially referred to as a “pump assembly”) configured to supply reduced or negative pressure. The wound cover 106 can be in fluidic communication with the wound 104.

With reference to FIG. 1B, the conduit 108 can have a bridge portion 130 that can have a proximal end portion and a distal end portion (the distal end portion being closer to the wound 104 than the proximal end portion, and an applicator 132 at the distal end of the bridge portion 130 forming the flexible suction adapter (or conduit) 108. A connector 134 can be disposed at the proximal end of the bridge portion 130, so as to connect to at least one of the channels that can extend along a length of the bridge portion 130 of the conduit 108 shown in FIG. 1B. A cap 140 can be coupled with a portion of the conduit 108 and can, in some cases, as illustrated, be attached to the connector 134. The cap 140 can be useful in preventing fluids from leaking out of the proximal end of the bridge portion 130. The conduit 108 can be a Soft Port manufactured by Smith & Nephew. As mentioned, the negative pressure wound treatment system 100 can include a source of negative pressure, such as the device 110, capable of supplying negative pressure to the wound 104 through the conduit 108. Though not required, the device 110 can also include a canister or other container for the storage of wound exudates and other fluids that can be removed from the wound.

The device 110 can be connected to the connector 134 via a conduit or tube 142. In use, the applicator 132 can be placed over an aperture formed in a cover 106 that is placed over a suitably-prepared wound or wound 104. Subsequently, with the wound therapy device 110 connected via the tube 142 to the connector 134, the wound therapy device 110 can be activated to supply negative pressure to the wound. Application of negative pressure can be applied until a desired level of healing of the wound is achieved.

The bridge portion 130 can comprise an upper channel material or layer positioned between an upper layer and an intermediate layer, with a lower channel material or layer positioned between the intermediate layer and a bottom layer. The upper, intermediate, and lower layers can have elongate portions extending between proximal and distal ends and can include a material that is fluid-impermeable, for example polymers such as polyurethane. It will of course be appreciated that the upper, intermediate, and lower layers can each be constructed from different materials, including semi-permeable materials. In some cases, one or more of the upper, intermediate, and lower layers can be at least partially transparent. In some instances, the upper and lower layers can be curved, rounded or outwardly convex over a majority of their lengths.

The upper and lower channel layers can be elongate layers extending from the proximal end to the distal end of the bridge 130 and can each preferably comprise a porous material, including for example open-celled foams such as polyethylene or polyurethane. In some cases, one or more of the upper and lower channel layers can be comprised of a fabric, for example a knitted or woven spacer fabric (such as a knitted polyester 3D fabric, Baltex 7970®, or Gehring 879®) or a nonwoven material, or terry-woven or loop-pile materials. The fibers may not necessarily be woven, and can include felted and flocked (including materials such as Flotex®) fibrous materials. The materials selected are preferably suited to channeling wound exudate away from the wound and for transmitting negative pressure or vented air to the wound site, and can also confer a degree of kinking or occlusion resistance to the channel layers. In one example, the upper channel layer can include an open-celled foam such as polyurethane, and the lower channel layer can include a fabric. In another example, the upper channel layer is optional, and the system can instead be provided with an open upper channel. The upper channel layer can have a curved, rounded or upwardly convex upper surface and a substantially flat lower surface, and the lower channel layer can have a curved, rounded or downwardly convex lower surface and a substantially flat upper surface.

The fabric or material of any components of the bridge 130 can have a three-dimensional (3D) structure, where one or more types of fibers form a structure where the fibers extend in all three dimensions. Such a fabric can in some cases aid in wicking, transporting fluid or transmitting negative pressure. In some cases, the fabric or materials of the channels can include several layers of material stacked or layered over each other, which can in some cases be useful in preventing the channel from collapsing under the application of negative pressure. The materials used in some implementations of the conduit 108 can be conformable and pliable, which can, in some cases, help to avoid pressure ulcers and other complications which can result from a wound treatment system being pressed against the skin of a patient.

The distal ends of the upper, intermediate, and lower layers and the channel layers can be enlarged at their distal ends (to be placed over a wound site), and can form a “teardrop” or other enlarged shape. The distal ends of at least the upper, intermediate, and lower layers and the channel layers can also be provided with at least one through aperture. This aperture can be useful not only for the drainage of wound exudate and for applying negative pressure to the wound, but also during manufacturing of the device, as these apertures can be used to align these respective layers appropriately.

In some implementations, a controlled gas leak 146 (sometimes referred to as gas leak, air leak, or controlled air leak) can be disposed on the bridge portion 130, for example at the proximal end thereof. This air leak 146 can comprise an opening or channel extending through the upper layer of the bridge portion 130, such that the air leak 146 is in fluidic communication with the upper channel of the bridge portion 130. Upon the application of suction to the conduit 108, gas (such, as air) can enter through the gas leak 146 and move from the proximal end of the bridge portion 130 to the distal end of the bridge portion along the upper channel of the bridge portion 130. The gas can then be suctioned into the lower channel of the bridge portion 130 by passing through the apertures through the distal ends of the upper, intermediate, and lower layers.

The air leak 146 can include a filter. Preferably, the air leak 146 is located at the proximal end of the bridge portion 130 so as to minimize the likelihood of wound exudate or other fluids coming into contact and possibly occluding or interfering with the air leak 146 or the filter. In some instances, the filter can be a microporous membrane capable of excluding microorganisms and bacteria, and which may be able to filter out particles larger than 45 μm. Preferably, the filter can exclude particles larger than 1.0 μm, and more preferably, particles larger than 0.2 μm. Advantageously, some implementations can provide for a filter that is at least partially chemically-resistant, for example to water, common household liquids such as shampoos, and other surfactants. In some cases, reapplication of vacuum to the suction adapter or wiping of the exposed outer portion of the filter may be sufficient to clear any foreign substance occluding the filter. The filter can be composed of a suitably-resistant polymer such as acrylic, polyethersulfone, or polytetrafluoroethylene, and can be oleophobic or hydrophobic. In some cases, the gas leak 146 can supply a relatively constant gas flow that does not appreciably increase as additional negative pressure is applied to the conduit 108. In instances of the negative pressure wound treatment system 100 where the gas flow through the gas leak 146 increases as additional negative pressure is applied, preferably this increased gas flow will be minimized and not increase in proportion to the negative pressure applied thereto. Further description of such bridges, conduits, air leaks, and other components, features, and details that can be used with any implementations of the negative pressure wound treatment systems disclosed herein are found in U.S. Pat. No. 8,801,685, which is incorporated by reference in its entirety as if fully set forth herein.

Any of the wound therapy devices (such as, the device 110 or 110′) disclosed herein can provide continuous or intermittent negative pressure therapy. Continuous therapy can be delivered at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. Intermittent therapy can be delivered between low and high negative pressure set points (sometimes referred to as setpoint). Low set point can be set at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, or below −180 mmHg. High set point can be set at above-25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. During intermittent therapy, negative pressure at low set point can be delivered for a first time duration, and upon expiration of the first time duration, negative pressure at high set point can be delivered for a second time duration. Upon expiration of the second time duration, negative pressure at low set point can be delivered. The first and second time durations can be same or different values.

In operation, the wound filler 102 can be inserted into the cavity of the wound 104, and wound cover 106 can be placed so as to seal the wound 104. The wound therapy device 110′ can provide negative pressure to the wound cover 106, which can be transmitted to the wound 104 via the wound filler 102. Fluid (such as, wound exudate) can be drawn through the conduit 108′ and stored in a canister. In some cases, fluid is absorbed by the wound filler 102 or one or more absorbent layers (not shown).

Wound dressings that can be utilized with the pump assembly and systems of the present application include Renasys-F, Renasys-G, Renasys AB, and Pico Dressings available from Smith & Nephew. Further description of such wound dressings and other components of a negative pressure wound therapy system that can be used with the pump assembly and systems of the present application are found in U.S. Patent Publication Nos. 2012/0116334, 2011/0213287, 2011/0282309, 2012/0136325, U.S. Pat. No. 9,084,845, and International App. No. PCT/EP2020/078376, each of which is incorporated by reference in its entirety as if fully set forth herein. In some cases, other suitable wound dressings can be utilized.

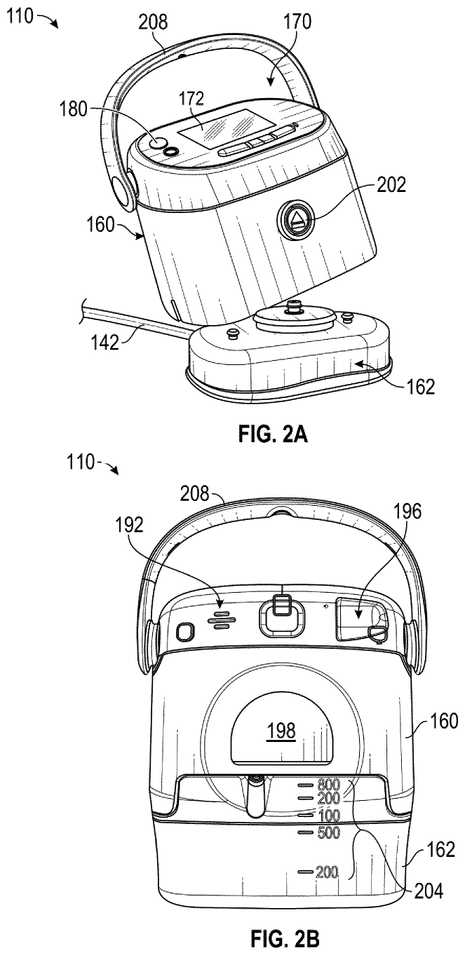

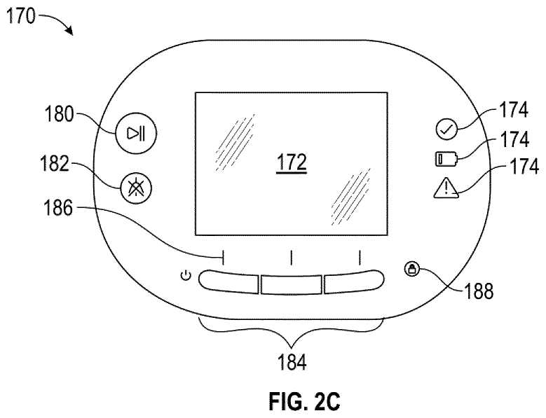

FIGS. 2A-2C show the negative pressure wound therapy device 110. As illustrated, a pump assembly 160 and canister 162 can be connected, thereby forming the wound therapy device 110. With reference to FIG. 2C, the pump assembly 160 can include an interface panel 170 having a display 172, one or more indicators 174, or one or more controls or buttons, including, for example and without limitation, a therapy start and pause button 180 or an alarm/alert mute button 182. The interface panel 170 can have one or more input controls or buttons 184 (three being shown) that can be used to control any functions of the pump assembly 160 or the interface panel 170. For example and without limitation, one or more of the buttons 184 can be used to turn the pump assembly 160 on or off, to start or pause therapy, to operate and monitor the operation of the pump assembly 160, to scroll through menus displayed on the display 172, or to control or perform other functions. In some cases, the command buttons 184 can be programmable, and can be made from a tactile, soft rubber.

Additionally, the interface panel 170 can have visual indicators 186 that can indicate which of the one or more buttons 184 is active. The interface panel 170 can also have a lock/unlock control or button 188 that can be configured to selectively lock or unlock the functionality of the various buttons (e.g., buttons 184) or the display 172. For example, therapy setting adjustment can be locked/unlocked via the lock/unlock control 188. When the lock/unlock button 188 is in the locked state, depressing one or more of the various other buttons or the display will not cause the pump assembly 160 to change any display functions or performance functions of the device. This way, the interface panel 170 will be protected from inadvertent bumping or touching of the various buttons or display. The interface panel 170 can be located on an upper portion of the pump assembly 160, for example and without limitation on an upward facing surface of the pump assembly 160.

The display 172, which can be a screen such as an LCD screen, can be mounted in a middle portion of the interface panel 170. The display 172 can be a touch screen display. The display 172 can support playback of audiovisual (AV) content, such as instructional videos, and render a number of screens or graphical user interfaces (GUIs) for configuring, controlling, and monitoring the operation of the pump assembly 160.

The one or more indicators 174 can be lights (such as, LEDs) and can be configured to provide a visual indication of alarm conditions and or a status of the pump. For example and without limitation, the one or more indicators 174 can be configured to provide a visual indication of a status of the pump assembly 160 or other components of the negative pressure wound treatment system 100, including without limitation the conduit 108 or the wound cover 106 (such as, to provide an indication of normal operation, low battery, a leak, canister full, blockage, overpressure, or the like). Any one or more suitable indicators can be additionally or alternatively used, such as visual, audio, tactile indicator, and so on.

FIG. 2B shows a back or rear view of the wound therapy device 110 shown in the FIG. 2A. As shown, the pump assembly 160 can include a speaker 192 for producing sound. For example and without limitation, the speaker 192 can generate an acoustic alarm in response to deviations in therapy delivery, non-compliance with therapy delivery, or any other similar or suitable conditions or combinations thereof. The speaker 192 can provide audio to accompany one or more instructional videos that can be displayed on the display 172.

The pump assembly 160 can be configured to provide easy access (such as, an access door on the casing of the pump assembly) to one or more filters of the pump assembly 160, such as antibacterial filters. This can enable a user (such as, a healthcare provider or patient) to more easily access, inspect or replace such filters. The pump assembly 160 can also include a power jack 196 for providing power to the pump assembly 160 or for charging and recharging an internal power source (such as, a battery). Some implementations of the pump assembly 160 can include a disposable or renewable power source, such as one or more batteries, so that no power jack is needed. The pump assembly 160 can have a recess 198 formed therein to facilitate gripping of the pump assembly 160.

The canister 162 can hold fluid aspirated from the wound 104. For example, the canister 162 can have an 800 mL (or approximately 800 mL) capacity, or from a 300 mL or less capacity to a 1000 mL or more capacity, or any capacity level in this range. The canister 162 can include a tubing for connecting to the conduit 108 in order to form a fluid flow path. The canister 162 can be replaced with another canister, such as when the canister 162 has been filled with fluid. With reference to FIG. 2A, the wound therapy device 110 can include a canister inlet tube 142 (also referred to herein as a dressing port connector) in fluid communication with the canister 162. For example and without limitation, the canister inlet tube 142 can be used to connect with the conduit 108.

The canister 162 can be selectively coupleable and removable from the pump assembly 160. With reference to FIG. 2A, in some cases, a canister release button 202 can be configured to selectively release the canister 162 from the pump assembly 160. With reference to FIG. 2B, the canister 162 can have one or more fill lines or graduations 204 to indicate to the user and amount of fluid or exudate stored within the canister 162. The wound therapy device 110 can have a handle 208 that can be used to lift or carry the wound therapy device 110. The handle 208 can be coupled with the pump assembly 160 and can be rotatable relative to the wound therapy device 110 so that the handle can be rotated upward for lifting or carrying the wound therapy device 110 or the pump assembly 160, or rotated into a lower profile in a more compact position when the handle is not being used. In some cases, the handle 208 can be coupled with the pump assembly 160 in a fixed position. The handle 208 can be coupled with an upper portion of the pump assembly 160 or can be removable from the wound therapy device 110.

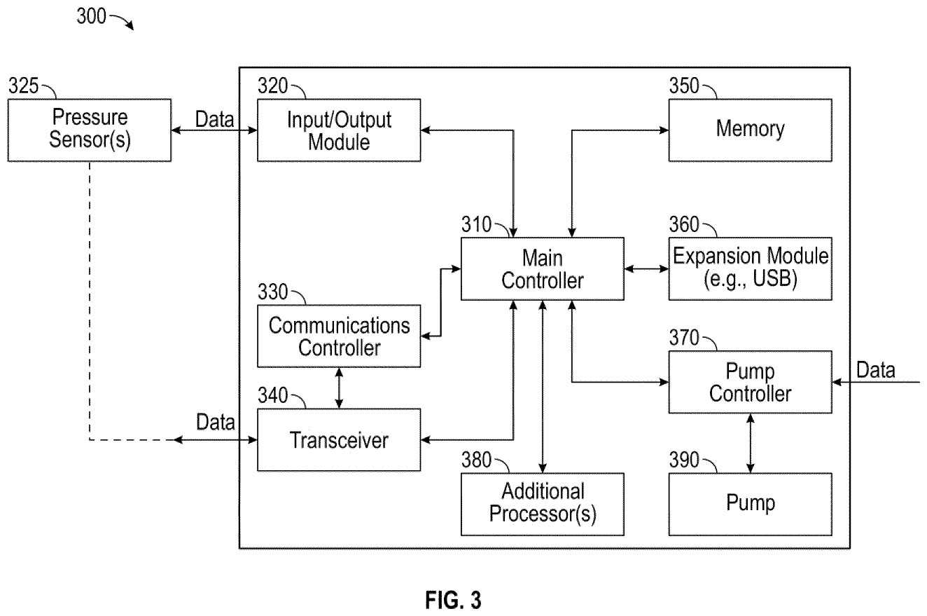

FIG. 3 illustrates a schematic of a control system 300 that can be employed in any of the wound therapy devices described herein, such as in the wound therapy device 110. Electrical components can operate to accept user input, provide output to the user, operate the pressure source, provide connectivity, and so on. A first processor (such as, a main controller 310) can be responsible for user activity, and a second processor (such as, a pump controller 370) can be responsible for controlling another device, such as a pump 390.

An input/output (I/O) module 320 can be used to control an input and/or output to another component or device, such as the pump 390, one or more sensors (for example, one or more pressure sensors 325 configured to monitor pressure in one or more locations of the fluid flow path), or the like. For example, the I/O module can receive data from one or more sensors through one or more ports, such as serial (for example, I2C), parallel, hybrid ports, and the like. Any of the pressure sensors can be part of the wound therapy device or the canister. In some cases, any of the pressure sensors 325 can be remote to the wound therapy device, such as positioned at or near the wound (for example, in the dressing or the conduit connecting the dressing to the wound therapy device). In such implementations, any of the remote pressure sensors can communicate with the I/O module over a wired connection or with one or more transceivers 340 over a wireless connection.

The main controller 310 can receive data from and provide data to one or more expansion modules 360, such as one or more USB ports, SD ports, Compact Disc (CD) drives, DVD drives, FireWire ports, Thunderbolt ports, PCI Express ports, and the like. The main controller 310, along with other controllers or processors, can store data in memory 350 (such as one or more memory modules), which can be internal or external to the main controller 310. Any suitable type of memory can be used, including volatile or non-volatile memory, such as RAM, ROM, magnetic memory, solid-state memory, Magnetoresistive random-access memory (MRAM), and the like.

The main controller 310 can be a general purpose controller, such as a low-power processor or an application specific processor. The main controller 310 can be configured as a “central” processor in the electronic architecture of the control system 300, and the main controller 310 can coordinate the activity of other processors, such as the pump controller 370, one or more communications controllers 330, and one or more additional processors 380. The main controller 310 can run a suitable operating system, such as a Linux, Windows CE, VxWorks, etc.

The pump controller 370 can control the operation of a pump 390, which can generate negative or reduced pressure. The pump 390 can be a suitable pump, such as a diaphragm pump, peristaltic pump, rotary pump, rotary vane pump, scroll pump, screw pump, liquid ring pump, diaphragm pump operated by a piezoelectric transducer, voice coil pump, and the like. The pump controller 370 can measure pressure in a fluid flow path, using data received from one or more pressure sensors 325, calculate the rate of fluid flow, and control the pump. The pump controller 370 can control the pump actuator (such as, a motor) so that a desired level of negative pressure is achieved in the wound 104. The desired level of negative pressure can be pressure set or selected by the user. The pump controller 370 can control the pump (for example, pump motor) using pulse-width modulation (PWM) or pulsed control. A control signal for driving the pump can be a 0-100% duty cycle PWM signal. The pump controller 370 can perform flow rate calculations and detect alarms. The pump controller 370 can communicate information to the main controller 310. The pump controller 370 can be a low-power processor.

Any of the one or more communications controllers 330 can provide connectivity (such as, a wired or wireless connection 332). The one or more communications controllers 330 can utilize one or more transceivers 340 for sending and receiving data. The one or more transceivers 340 can include one or more antennas, optical sensors, optical transmitters, vibration motors or transducers, vibration sensors, acoustic sensors, ultrasound sensors, or the like. Any of the one or more transceivers 340 can function as a communications controller. In such case, the one or more communications controllers 330 can be omitted. Any of the one or more transceivers 340 can be connected to one or more antennas that facilitate wireless communication. The one or more communications controllers 330 can provide one or more of the following types of connections: Global Positioning System (GPS), cellular connectivity (for example, 2G, 3G, LTE, 4G, 5G, or the like), NFC, Bluetooth connectivity (or BLE), radio frequency identification (RFID), wireless local area network (WLAN), wireless personal area network (WPAN), WiFi connectivity, Internet connectivity, optical connectivity (for example, using infrared light, barcodes, such as QR codes, etc.), acoustic connectivity, ultrasound connectivity, or the like. Connectivity can be used for various activities, such as pump assembly location tracking, asset tracking, compliance monitoring, remote selection, uploading of logs, alarms, and other operational data, and adjustment of therapy settings, upgrading of software or firmware, pairing, and the like.

Any of the one or more communications controllers 330 can provide dual GPS/cellular functionality. Cellular functionality can, for example, be 3G, 4G, or 5G functionality. The one or more communications controllers 330 can communicate information to the main controller 310. Any of the one or more communications controllers 330 can include internal memory or can utilize memory 350. Any of the one or more communications controllers 330 can be a low-power processor.

The control system 300 can store data, such as GPS data, therapy data, device data, and event data. This data can be stored, for example, in memory 350. This data can include patient data collected by one or more sensors. The control system 300 can track and log therapy and other operational data. Such data can be stored, for example, in the memory 350.

Using the connectivity provided by the one or more communications controllers 330, the control system 300 can upload any of the data stored, maintained, or tracked by the control system 300 to a remote computing device, such as the device 334. The control system 300 can also download various operational data, such as therapy selection and parameters, firmware and software patches and upgrades, and the like (for example, via the connection to the device 334). The one or more additional processors 380, such as processor for controlling one or more user interfaces (such as, one or more displays), can be utilized. In some cases, any of the illustrated or described components of the control system 300 can be omitted depending on an embodiment of a wound monitoring or treatment system in which the control system 300 is used.

Any of the negative pressure wound therapy devices described herein can include one or more features disclosed in U.S. Pat. No. 9,737,649 or U.S. Patent Publication No. 2017/0216501, each of which is incorporated by reference in its entirety.

### Multiple Dressing Negative Wound Therapy

FIG. 4 illustrates another negative pressure wound treatment system 400. The system 400 can include a wound therapy device capable of supplying negative pressure to the wound site or sites, such as wound therapy device 110. The wound therapy device 110 can be in fluidic communication with one or more wound dressings 406a, 406b (collectively referred to as 406) so as to supply negative pressure to one or more wounds, such as the wounds 104a and 104b. A first fluid flow path can include components providing fluidic connection from the wound therapy device 110 to the first wound dressing 406a. As a non-limiting example, the first fluid flow path can include the path from the wound dressing 406a to the wound therapy device 110 or the path from the first wound dressing 406a to an inlet 446 of a branching attachment (or connector) 444 in fluidic connection with the wound therapy device 110. Similarly, a second fluid flow path can include components providing fluidic connection from the wound therapy device 110 to the second wound dressing 406b.

The system 400 can be similar to the system 100 with the exception that multiple wounds 104a and 140b are being treated by the system 400. The system 400 can include any one or more of the components of the system 100, which are illustrated in FIG. 4 with appended letter “a” or “b” to distinguish between the first and second wounds (such as, the wounds 104a and 104b, the covers 106a and 106b). As illustrated, the system 400 can include a plurality of wound dressings 406a, 406b (and corresponding fluid flow paths) in fluidic communication with the wound therapy device 110 via a plurality of suction adapters, such as the adapter 108. The suction adapters can include any one or more of the components of the adapter 108, which are illustrated in FIG. 4 with appended letter “a” or “b” to distinguish between the first and second wounds (such as, the bridge portions 130a and 130b, the connectors 134a and 134b, and the caps 140a and 140b).

The wound therapy device 110 can be fluidically coupled via the tube 142 with the inlet 446 of the connector 444. The connector 444 can be fluidically coupled via branches 445a, 445b and tubes or conduits 442a, 442b with the connectors 134a, 134b, which can be fluidically coupled with the tubes or conduits 130a, 130b. The tubes or conduits 130a, 130b can be fluidically coupled with the dressings 406a, 406b. Once all conduits and dressing components are coupled and operably positioned, the wound therapy device 110 can be activated, thereby supplying negative pressure via the fluid flow paths to the wounds 104a, 104b. Application of negative pressure can be applied until a desired level of healing of the wounds 104a, 104b is achieved. Although two wounds and wound dressing are illustrated in FIG. 4, some implementations of the wound therapy device 110 can provide treatment to a single wound (for instance, by closing the unused branch 445a or 445b of the connector 444) or to more than two wounds (for instance, by adding branches to the connector 444).

The system 400 can include one or more features disclosed in U.S. Patent Publication No. 2020/0069850 or International Publication No. WO2018/167199, each of which is incorporated by reference in its entirety.

### Canisterless Pump Assembly

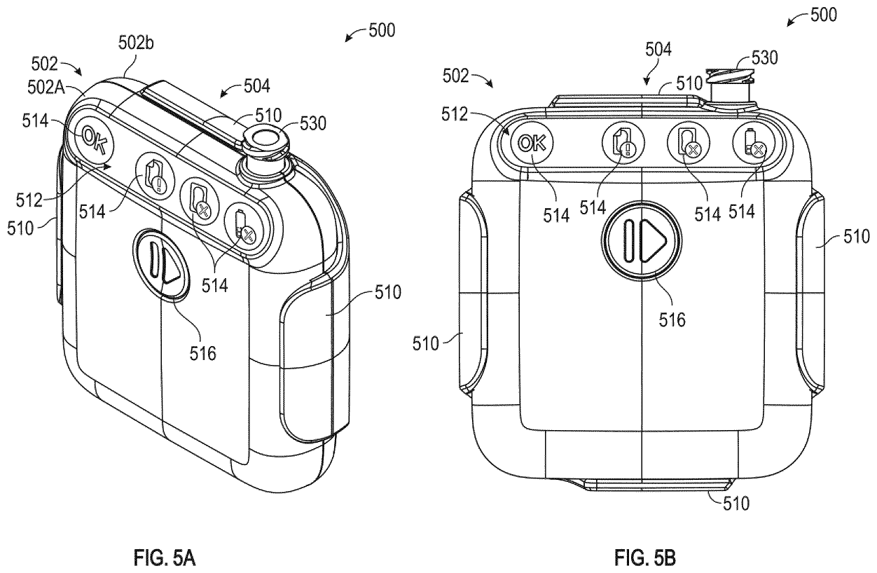

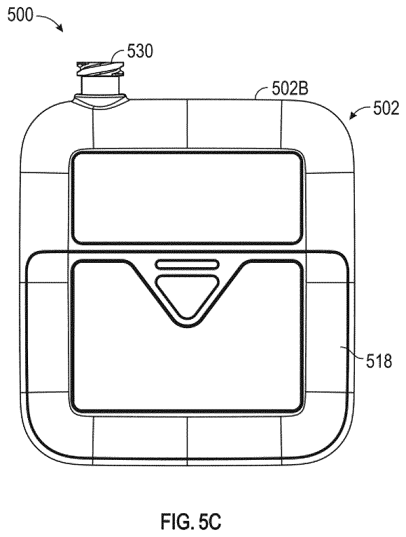

FIGS. 5A, 5B, and 5C illustrate perspective, front, and rear views of a negative pressure wound therapy device 500 (sometimes referred to as a wound therapy device). The wound therapy device 500 can include a housing 502 and a mounting component 510 (such as an attachment). The mounting component 510 can be removably attached to the housing 502, such that the wound therapy device 500 can be used with or without the mounting component 510. For example, FIG. 5C illustrates the wound therapy device 500 without the mounting component 510. The mounting component 510 can be designed to allow the wound therapy device 500 to be mounted on another object such as, but not limited to, a user's person. The mounting component 510 can include a clip 504 designed to retain the mounting component 510 on a user's outerwear, such as on a user's pocket, a pouch, a belt, a flap, or otherwise.

The housing 502 (sometimes referred to as “outer housing”) can contain or support components of the wound therapy device 500. The housing 502 can be formed from one or more portions, such as a front portion 502A and a rear portion 502B, which can be removably attached to form the housing 502.

The housing 502 can include a user interface 512 which can be designed to provide a user with information (for example, information regarding an operational status of the wound therapy device 500). The user interface 512 can include one or more indicators, such as icons 514, which can alert the user to one or more operating or failure conditions of the reduced pressure wound therapy system.

The wound therapy device 500 can include one or more user input features, such as button 516, designed to receive an input from the user for controlling the operation of the wound therapy device 500. A single button can be present which can be used to activate and deactivate the reduced pressure wound therapy device or control other operating parameters of the wound therapy device 500.

The wound therapy device 500 can include a connector 530 for connecting a tube or conduit to the wound therapy device 500. The connector 530 can be used to connect the wound therapy device 500 to a wound dressing.

The wound therapy device 500 can be a canisterless device. The wound dressing can retain fluid (such as, exudate) aspirated from the wound. Such a dressing can include a filter, such as a hydrophobic filter, that prevents passage of liquids downstream of the wound dressing (toward the wound therapy device 500).

The wound therapy device 500 can include a cover 518, as illustrated in FIG. 5C and which can be removable. The cover 518 can cover a cavity (not shown) in which one or more power sources, such as batteries, for powering the wound therapy device 500 are positioned.

The wound therapy device 500 can include one or more controllers or other electronic components described herein, such as in connection with FIG. 3. The wound therapy device 500 can be similar to the Pico negative pressure wound therapy device manufactured by Smith & Nephew.

Any of the negative pressure wound therapy devices described herein can include one or more features disclosed in U.S. Patent Publication No. 2019/0231939, which is incorporated by reference in its entirety.

### Accelerating Delivery of Negative Pressure Wound Therapy

At the initiation of negative pressure wound therapy (or subsequent to a prolonged pause in the delivery of therapy), a negative pressure wound therapy device may need to evacuate a large amount of fluid (for instance, air) to establish a desired negative pressure set point at the wound. Particularly for larger wounds (which may need to be covered by a larger dressing), the period of time for establishing the desired negative pressure set point may be lengthy. In some cases, during initial pump down when the desired negative pressure set point is being established for the first time or following a period of inactivity of the device, the device may timeout responsive to the initial pump down taking too long. The timeout can be caused by the device possibly misinterpreting the delay in establishing the desired negative pressure set point as a leak condition in the fluid flow path and, as a result, deactivating the negative pressure source to conserve power, reduce noise, etc. Such operation is described in U.S. Pat. No. 8,905,985, which is incorporated by reference in its entirety.

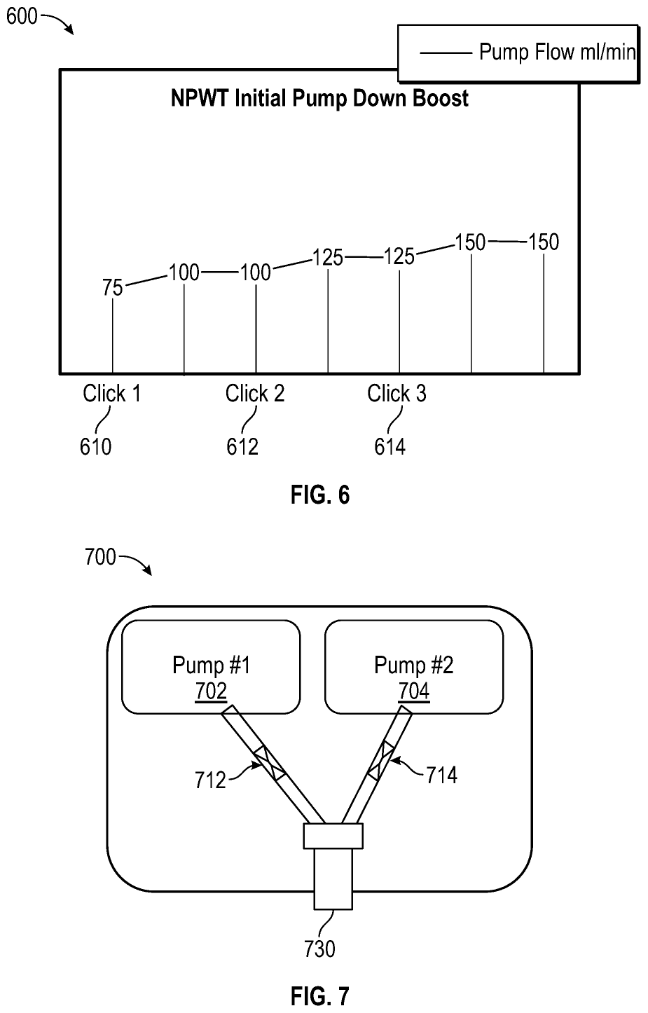

To achieve the desired negative pressure set point faster, the device can allow the user to increase the fluid flow generated by the negative pressure source. FIG. 6 illustrates different fluid flow rates in the graph 600. The user can be allowed to select fluid flow rates of, for instance, 75 ml/min, 100 ml/min, 125 ml/min, and 150 ml/min. In some cases, the user can select a desired flow rate by operating one or more user interface components of the device (such as, a switch or button). For example, with reference to the device 500, the user can operate the button 516. As another example, with reference to the device 110 described herein, the user can operate the button 180. The user can select the desired flow rate by clicking the button a desired number of times. With reference to FIG. 6, a default fluid flow rate of 75 ml/min for establishing the desired negative pressure set point can be selected by the user with a single click (or press) 610 of a button when initiating negative pressure wound therapy. The default flow rate can be selected to preserve capacity of the power source, reduce noise, reduce vibration, etc.

A subsequent (second) click 612 of the button can allow the user to select an increased fluid flow rate of 100 ml/min. A subsequent (third) click 614 of the button can allow the user to select an increased fluid flow rate of 125 ml/min. A subsequent (fourth) click of the button can allow the user to select a maximum flow rate of 150 ml/min. A controller of the device (which can be any of the controllers disclosed herein) can operate the negative pressure source to provide the increased fluid flow rate. In some implementations, a controller can overdrive the negative pressure source, for example, by providing an increased voltage, current, or duty cycle to drive an actuator of the negative pressure source and cause an increase in the fluid flow rate.

In some cases, the device's user interface can include a single switch or button. For instance, the device 500 can include a single button 516 configured to activate and deactivate provision of negative pressure wound therapy by the device. During the initial pump down, a first click of the button 516 can activate the negative pressure source to aspirate fluid from the wound at a default fluid flow rate (such as, 75 ml/min). Subsequent presses of the button 516 during the initial pump down can increase the fluid flow rate, as described herein. After the initial pump down has been completed and a desired negative pressure set point has been established at the wound, a press of the button 516 can pause provision of negative pressure wound therapy (for instance, by deactivating the negative pressure source). Alternate presses of the button 516 following the establishment of the desired negative pressure set point at the wound (or during maintenance of the desired negative pressure set point) can pause and restart negative pressure wound therapy. This operation is described in U.S. Pat. No. 8,905,985, which is incorporated by reference in its entirety.

In some implementations, different presses of the button 516 can cause the device to perform different actions. For instance, one or more short presses of the button 516 during the initial pump down can cause increase in the fluid flow rate provided by the negative pressure source. As another example, one or more long presses of the button 516 during the initial pump down can pause and restart of negative pressure wound therapy.

In some cases, a separate user interface component can be provided to the user to increase the fluid flow rate. Such separate user interface component can be a switch or a button. In some implementations, a user interface can be provided on a remote computing device, such as the device 334 (which can be a smartphone or a tablet. Indication(s) of the increase(s) in the fluid flow rate can be provided using any of the approaches disclosed herein. For instance, the indication(s) can be one or more of visual, audible, or tactile. For instance, a display can indicate progress toward the target negative pressure set point (such as, graphically or via a percentage).

Any of the negative pressure wound therapy devices can include multiple negative pressure sources. With reference to FIG. 7, a negative pressure wound therapy device 700 that can be similar to the device 500 or 110. The device 700 can include a first negative pressure source 702 connected via a one-way valve 712 (or non-return valve) to a connector 730, which can be similar to the connector 530 and used to connect the device 700 to a wound dressing. The device 700 can include a second negative pressure source 704 connected via a one-way valve 714 to the connector 730. The first negative pressure source 702 can be configured to provide a default fluid flow rate for aspirating fluid from the wound. The second negative pressure source 704 can be configured to be inactivate until the user has selected a fluid flow rate that in greater than the default fluid flow rate. The second negative pressure source 704 can assist the first negative pressure source 702 with aspirating fluid from the wound during the initial pump down. After the initial pump down has concluded and a desired negative pressure set point has been established at the wound, the second negative pressure source 704 can be deactivated.

The approaches described in this section can be performed under control of one or more controllers of the negative pressure wound therapy device.

The approaches described in this section can advantageously provide the flexibility for effectively treating wounds of different sizes. A better seal and quicker establishment of desired negative pressure at the wound can be achieved.

### Reducing Rate of Delivering Negative Pressure Wound Therapy

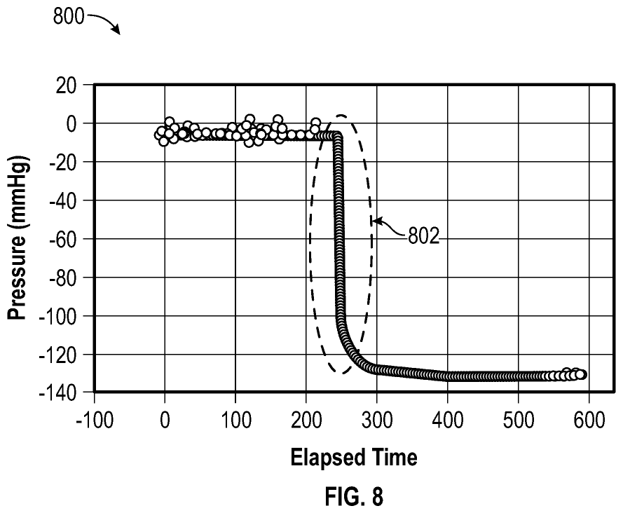

FIG. 8 illustrates a plot 800 for reducing pressure at the wound to a desired negative pressure set point (such as, about −120 mmHg). The plot 800 illustrates an initial pump down segment 802 (such as, from 0 mmHg to −120 mmHg). In some cases, a patient can experience significant pain during the sharp pressure reduction as a result of the initial pump down illustrated in FIG. 8. Accordingly, it can be advantageous to manage the rate of the initial pump down to promote patient comfort.

A negative pressure wound therapy device (which can be any of the devices described herein, such as 110 or 500) can be configured to provide the user with the ability to manage the rate of reducing negative pressure during the initial pump down. In other words, the user can configure one or more of the slope, gradient, or shape of the initial pump down segment 802. This can be accomplished by selecting an appropriate compression rate for reducing the pressure, as described in described in U.S. Pat. No. 10,744,239, which is incorporated by reference in its entirety.

Another approach for controlling the rate of reducing negative pressure is to allow the user to control the rate via one or more user interface components of the device (such as, a switch or button). For example, with reference to the device 500, the user can operate the button 516. As another example, with reference to the device 110 described herein, the user can operate the button 180. The user can control the rate by pausing provision of negative pressure. For instance, the user can pause provision of therapy by pressing or holding down the button. In some cases, the user interface component for controlling the rate of reducing negative pressure can be provided on a remote computing device, such as the device 334 (for example, as smartphone or a tablet).

Pressing or holding the button (or operating the button or another user interface component in another way) can cause the device to trigger a rest period by deactivating the negative pressure source or decreasing the fluid flow rate or negative pressure level provided by the negative pressure source. If the button is not pressed or held (or the button or another user interface component in not operated in another way), the device can continue to ramp down the pressure at a default rate (for instance, between 0.1 mmHg/sec and 50 mmHg/sec). Pressing or holding the button (or operating the button or another user interface component in another way) can halt the ramp down of pressure. In some cases, pressing or holding the button can alternatively continue the ramp down of pressure, while releasing the button can pause the ramp down.

In some implementations, the device can monitor the duration of time during which delivery of negative pressure has been paused. Such monitoring can be performed using a timer. Responsive to a determination that the duration of time satisfies a threshold and the target negative pressure set point has not been established, the device can activate the negative pressure source to resume the ramp down of pressure irrespective of the user pressing or holding the button (or operating the button or another user interface control in some other way). The device can disable the button (or another user interface control) responsive to the determination.

In some cases, a rotary switch (or a rotary knob) can be used by the user to set the desired pressure ramp down rate. The rate can be proportional to the rotary switch position. As described above, a duration of time can be monitored such that, if the negative pressure set point has not been reached after a threshold duration, the device can ramp down the pressure at the default rate. The device can disable the rotary switch.

In some cases, the user can utilize one or more user interface components of the device (such as, a switch or button) to control the target negative pressure set point. For example, the user may initially select −40 mmHg as the target negative pressure set point. After −40 mmHg has been established at the wound, the user may increase the target negative pressure set point to −60 mmHg. This process can continue until therapeutic pressure has been established at the wound. Such approach can be advantageous when using traditional negative pressure wound therapy dressings, since at some point during the depressurization of the wound, the wound tissue starts to contract under the vacuum. Because such contraction can be quite painful, it can be beneficial to provide the user with maximum control through this transition point.

Progress toward the target negative pressure set point can be provided via one or more indications (such as one or more of visual, audio, or tactile indications). For instance, a display could indicate progress toward the target negative pressure set point (such as, graphically or via a percentage).

The approaches described in this section can be performed under control of one or more controllers of the negative pressure wound therapy device.

### Other Variations

Although some embodiments describe negative pressure wound therapy, the systems, devices, and/or methods disclosed herein can be applied to other types of therapies usable standalone or in addition to TNP therapy. Systems, devices, and/or methods disclosed herein can be extended to any medical device, and in particular any wound monitoring and/or treatment device. For example, systems, devices, and/or methods disclosed herein can be used with devices that provide one or more of ultrasound therapy, oxygen therapy, neurostimulation, microwave therapy, active agents, antibiotics, antimicrobials, or the like. Such devices can in addition provide TNP therapy. As another example, systems, devices, and/or methods disclosed herein can be used with a wound debridement system, patient monitoring system, or the like. The systems and methods disclosed herein are not limited to medical devices and can be utilized by any electronic device.

Any of transmission of data described herein can be performed securely. For example, one or more of encryption, https protocol, secure VPN connection, error checking, confirmation of delivery, or the like can be utilized.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures or described herein may be implemented as software and/or firmware on a processor, controller,

ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1. A negative pressure wound therapy apparatus comprising:
a housing;
at least one negative pressure source supported by the housing, the at least one negative pressure source configured to aspirate, via a fluid flow path, fluid from a wound covered by a wound dressing;
a switch positioned on an exterior surface of the housing; and
a controller configured to operate the at least one negative pressure source, the controller further configured to:
responsive to receiving a first user input via the switch, operate the at least one negative pressure source to aspirate fluid from the wound at a first fluid flow rate; and
responsive to receiving, via the switch, a second user input subsequent to receiving the first user input, operate the at least one negative pressure source to aspirate fluid from the wound at a second fluid flow rate greater than the first fluid flow rate.

2. The negative pressure wound therapy apparatus of claim 1, wherein the switch is the only switch positioned on the exterior surface of the housing and configured to activate and deactivate provision of negative pressure wound therapy.

3. The negative pressure wound therapy apparatus of claim 1, wherein the controller is further configured to:
responsive to receiving a third user input via the switch subsequent to receiving the second user input, operate the at least one negative pressure source to aspirate fluid from the wound at a third fluid flow rate greater than the second fluid flow rate.

4. The negative pressure wound therapy apparatus of claim 3, wherein the controller is configured to operate the at least one negative pressure source to aspirate the fluid at the first, second, or third fluid flow rate during establishing a target negative pressure level at the wound.

5. The negative pressure wound therapy apparatus of claim 1, wherein the controller is configured to, subsequent to establishing a target negative pressure level at the wound:
responsive to receiving a fourth user input via the switch, deactivate the at least one negative pressure source to pause aspiration of fluid from the wound.

6. The negative pressure wound therapy apparatus of claim 3, wherein at least one negative pressure source comprises an actuator, and wherein the controller is configured to overdrive the actuator to operate the at least one negative pressure source to aspirate fluid from the wound at the second or third fluid flow rate.

7. The negative pressure wound therapy apparatus of claim 1, wherein the first fluid flow rate corresponds to a default fluid flow rate for establishing a target negative pressure level at the wound following initiation of negative pressure wound therapy.

8. The negative pressure wound therapy apparatus of claim 3, wherein the at least one negative pressure source comprises first and second negative pressure sources, the first negative pressure source configured to aspirate fluid from the wound at the first fluid flow rate.

9. The negative pressure wound therapy apparatus of claim 8, wherein the controller is configured to activate the second negative pressure source responsive to the second or third user input to assist the first negative pressure source to aspirate fluid from the wound at the second or third fluid flow rate.

10. The negative pressure wound therapy apparatus of claim 1, wherein the switch comprises a button.

11. The negative pressure wound therapy apparatus of claim 1, further comprising another switch configured to activate and pause provision of negative pressure wound therapy.

12. The negative pressure wound therapy apparatus of claim 1, wherein the apparatus comprises a canisterless apparatus.

13. (canceled)

14. (canceled)

15. (canceled)

16. A negative pressure wound therapy apparatus comprising:
a housing;
a negative pressure source supported by the housing, the negative pressure source configured to provide, via a fluid flow path, negative pressure to a wound covered by a wound dressing; and
a controller configured to operate the negative pressure source, the controller further configured to:
responsive to receiving a first user input, operate the negative pressure source to provide negative pressure to the wound at a first negative pressure rate; and
responsive to receiving a second user input subsequent to the first user input, operate the negative pressure source to provide negative pressure to the wound at a second negative pressure rate lower than the first negative pressure rate.

17. The negative pressure wound therapy apparatus of claim 16, further comprising a switch positioned on an exterior surface of the housing, wherein the first and second user inputs are received via the switch.

18. The negative pressure wound therapy apparatus of claim 17, wherein the switch is the only switch positioned on the exterior surface of the housing and configured to activate and deactivate provision of negative pressure wound therapy.

19. The negative pressure wound therapy apparatus of claim 17, wherein the switch comprises a rotary switch configured to be set by a user to a position of a plurality of positions associated with a plurality of second negative pressure rates.

20. The negative pressure wound therapy apparatus of claim 16, wherein the controller is configured to operate the negative pressure source to provide negative pressure to the wound at the first negative pressure rate or the second negative pressure rate during establishing a target negative pressure level at the wound.

21. The negative pressure wound therapy apparatus of claim 17, wherein the controller is further configured to:
responsive to a determination that 1) a duration of time during which the negative pressure source has been providing negative pressure to the wound at the second negative pressure rate satisfies a threshold and 2) a target negative pressure level has not been established at the wound, operate the negative pressure source to provide negative pressure to the wound at the first negative pressure rate.

22. The negative pressure wound therapy apparatus of claim 21, wherein the controller is further configured to disable the switch responsive to the determination.

23. The negative pressure wound therapy apparatus of claim 16, wherein the first negative pressure rate corresponds to a default negative pressure rate for establishing a target negative pressure level at the wound following initiation of negative pressure wound therapy.

24. (canceled)

25. (canceled)
